(19) United States
(12) Patent Application Publication
(10) Pub. No.: US 2007/0038306 A1
(43) Pub. Date: Feb. 15, 2007

(54) O'Gara femur prosthesis

(76) Inventor: Tadhg James O'Gara, Lynbrook, NY (US)

Correspondence Address: TADHG O'GARA, 199 WEBSTER STREET, WEST NEWTON, MA, 02465, US

(21) Appl. No.: 11/278,604

(22) Filed: Apr. 4, 2006

Related U.S. Application Data
(60) Provisional application No. 60/668,184, filed on Apr. 5, 2005.

(51) Int. Cl. A61F 2/36; A61F 2/38

(52) U.S. Cl. 623/22.42; 623/20.36; 623/22.46

(57) ABSTRACT

This patent describes a device to replace the proximal femoral head and neck. The uniqueness of the device is in its proximal design which incorporates a transverse tension bar which prevents failure of the femoral neck portion of the device in tension. This design characteristic permits the use of thinner materials which more closely resemble the elastic nature of bone itself while at the same time endowing the prosthesis with strength and resistance to failure. The device is designed to have a long intramedullary portion which would load the femur throughout its length as well as a modular attachment on its distal end for the insertion of a future knee replacement.

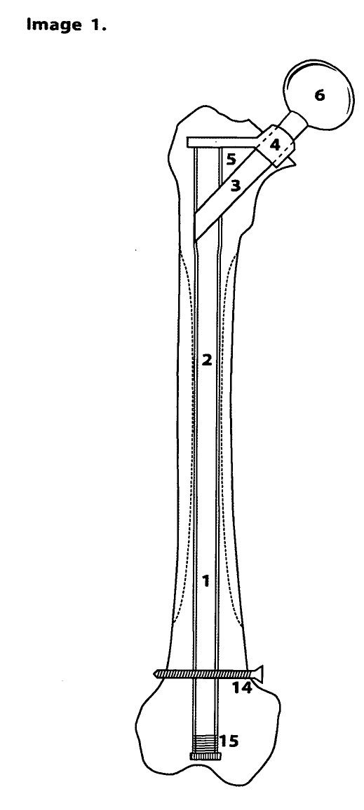

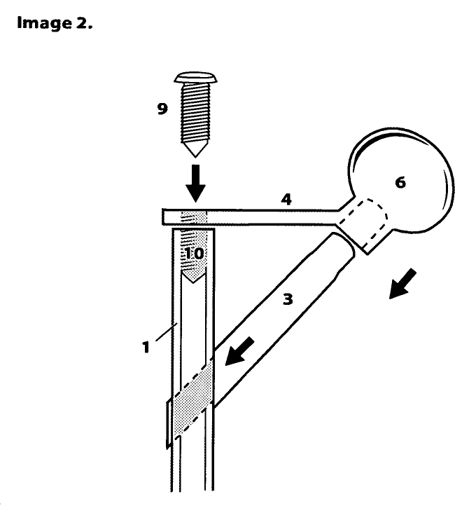

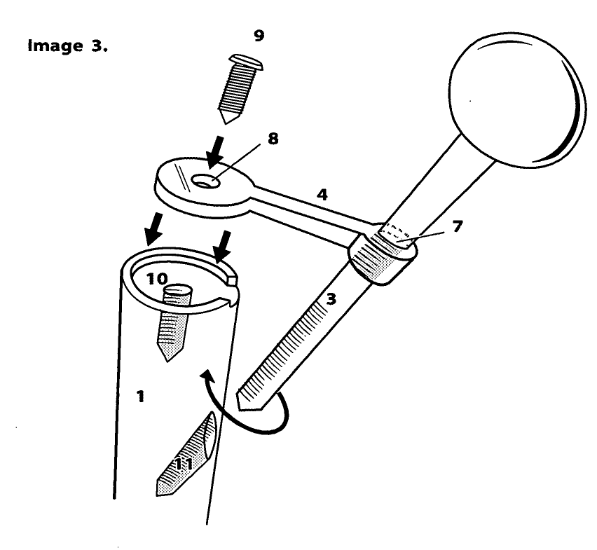

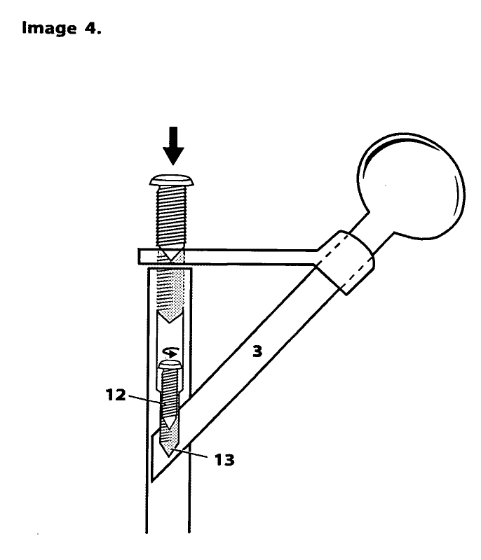

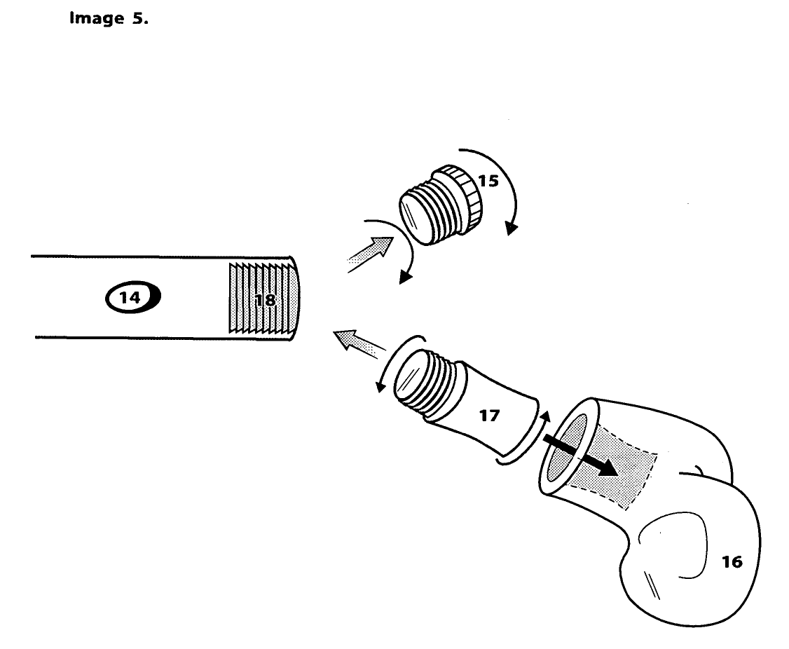

### BACKGROUND OF THE INVENTION

The subject invention relates to hip implants, particularly those that replace the head of the native femur. In the field of orthopaedic surgery, replacement of the native femoral head and neck is a common operation. The usual indications are for either fracture of the femoral neck or for degenerative diseases involving the femoral head.

The long term results of the surgical replacement of the proximal part of the femur are often hampered by mechanical failure of the natural proximal femoral bones' ability to host the implant for prolonged periods of time. A critical characteristic of a hip prosthesis is the ability to transmit the stress of weightbearing directly to the host bone while maintaining a stable, durable construct. The human femoral neck sustains stresses several times the body's weight therefore a prosthesis needs to be very strong and durable. These characteristics mean that conventional prostheses are also relatively stiff which equates to an inability to gently transmit the stresses of the femoral neck over a long area of native femur. The stiff prosthesis will bear the majority of the body's weight and transfer the stress to distally in the femur to the point where there is the abrupt transition between the femur prosthesis and the natural bone. Not only does this shield the proximal femur from experiencing the normal physiologic stress of weight bearing but it also places abnormally high stresses on the small transition zone between implant and native bone.

### SUMMARY OF THE INVENTION

The described invention solves the problems evident in the traditional hip prosthesis. This invention is unique in that it describes a femoral prosthesis which binds and loads the natural femur over its entire length. This is accomplished by the invention consisting of modular parts, one being a long intramedullary segment of the prosthesis which extends the major length of the femur. Proximally, the high stresses of the femoral neck are born by another modular part, a unique component of the invention which involves a strut connecting the intramedullary portion of the prosthesis to the femoral neck portion of the prosthesis which is a third modular part. This design therefore transmits the incredibly high stresses of the femoral neck to the entire femur due to its flexibility yet the prosthesis is durable because of the unique triangular design proximally. Because of these unique characteristics, the invention also preserves proximal femoral bone, a benefit in cases where a revision of the prosthesis is necessary.

This patent covers the design and architectural components of the hip prosthesis invention described here. There are many compositions or elements of materials that could possibly be used to construct the described device however the actual architectural design of the components are unique. Also, the cross sectional profile of each component of said invention can be of many shapes and designs. It is therefore understood that any prosthesis which may or may not have variations in the cross sectional design of the components will be included within the scope of this invention, as long as the larger architectural structure of these various components are described or claimed characteristics of this invention. For example, in cross section, the femoral neck component of the prosthesis can be cylindrical, sided with either flat, concave or convex sides, star shaped, oval shaped, triangular shaped, etc.

The distal end of the femoral neck component is designed to articulate into a mating hole in the intramedullary portion of the femoral prosthesis. This articulation would occur at a point where the oblique axis of the native femoral neck intersects the longitudinal axis of the femur shaft. At the proximal aspect of the neck prosthesis is where the head would sit. The weight of the body would therefore be born downward to the intramedullary portion of the prosthesis. The femoral neck portion can be thin and the tendency of this construct to fail in compression and shear will be opposed by another unique component of the invention: a transverse tension bar which supports the neck at its proximal end, bearing the tension, naturally born by the superior part of the native femur. This tension bar articulates with the proximal end of the neck or head and then extends laterally and horizontally to articulate with the superior part of the intramedullary portion of the invention. The prosthesis thus has the appearance of a triangle in its proximal architecture.

The invention also covers alternative attachments and articulations between the prosthesis femoral head, the cross strut and the femoral neck portions. In one example, the femoral head could be incorporated as part of the neck, around which the cross strut would attach, in another example of the invention, the femoral head is separate, articulating with the neck and cross strut independently.

Distally on the intramedullary portion of the implant can be a hole through the prosthesis designed for an interlocking screw through the bone and prosthesis. Also distally on the intramedullary portion of the implant is an “end cap” which is screwed into place with the male portion of the screw on the end cap and the female portion of the screw threads contained within the intramedullary portion of the implant or visa versa. This can be used if the patient later needed a knee replacement where the femoral component of the total knee replacement could be screwed into the intramedullary portion of the hip prosthesis once the end cap was removed during the total knee replacement surgery.

### BRIEF DESCRIPTION OF THE SEVERAL VIEWS OF THE DRAWINGS

Images 1-5 are exemplary renditions of the described invention. Image 1 is a frontal plane view of the invention's entire length. Images 2,3 and 4 are views of the various modifications made to the proximal (part nearer the head or hip joint) part of the invention included within the scope of this patent. Image 5 is an exemplary rendition of the distal (part farthest from the hip joint, toward the knee joint) part of the invention and includes a demonstration of the invention's ability to articulate with future knee implants.

### DETAILED DESCRIPTION OF THE INVENTION

An exemplary rendition of the invention is drawn in image 1. Note the long, intramedullary portion of the invention 1 which will bind the femur throughout its length, especially in the isthmus region seen as 2. The unique portion of the hip prosthesis is that proximally, the intramedullary portion 1 has articulations for two critical parts of the device. Firstly, the intramedullary portion 1 articulates with the femoral neck portion 3 which sustains compressive loads. Secondly, the intramedullary portion articulates at its most proximal end with a tension bar 4 which sustains the tension forces born by this region of the femoral neck. This critical and unique design characteristic allows the component parts to be thinner material, because the tendancy for a thin femoral neck 3 to fail in torsion is balanced by the tension bar 4's opposition of that tension moment. This same design characteristic also allows the preservation of a wedge shaped portion of femoral neck bone seen as 5.

Image 1 demonstrates an example where the femoral head 6 is a proximal continuation of the femoral neck 3. This feature is again shown in image 3, where the tension bar 4 articulates with this construct as the femoral head and neck construct insert through a matching cavity 7 in the tension bar's medial aspect.

Another example of a potential articulation is seen in image 2 where the femoral head 6 is a medial continuation of the tension bar 4. The proximal aspect of the femoral neck 3 articulates with the femoral head 6 via its insertion into a matching shaped cavity in the base of the femoral head 6.

Image 2 and Image 3 also demonstrates a potential articulation of the tension bar's lateral aspect 4 with the intramedullary rod 1 via a threaded screw hole 8 through which a screw 9 would lock the lateral aspect of the tension bar into the intramedullary rod which has a matching hole 10 to receive the screw.

Image 1 and 2 demonstrates a potential press fit articulation of the femoral neck 3 with the intramedullary rod 1. Image 3 demonstrates an exemplary variation of this articulation which could include a threaded screw hole 11 in the intramedullary portion into which the distal femoral neck 3 screwed into. Image 4 demonstrates another exemplary articulation of the femoral neck 3 with the shaft via a “set screw” 12 within the intramedullary portion which can be tightened down, locking the femoral neck portion 3 in place by advancing into a groove 13 within the femoral neck.

There is a transverse hole 14 distally seen in image 1 and in cross section in image 5 whereby interlocking bone screws or pegs can be placed to augment fixation of the prosthesis. Also seen in images 1 and 5 is an end cap 15 which can be removed at subsequent surgeries if a prosthesis needs to be inserted onto the distal femur for a total knee replacement's femoral part 16. As seen in image 5, the femur prosthesis for the total knee replacement would be press fit into an articulation 17 which screws into the threaded end 18 of the intramedullary portion of this invention.

What is claimed is:

1. A device for the replacement of the native femoral head and neck, consisting of an intramedullary portion, a femoral neck portion attached to the intramedullary portion at the distal juncture of the native neck and shaft, and a transverse cross strut connecting the proximal aspect of the intramedullary portion to the proximal aspect of the neck, at the junction of where the invention's replacement femur head sits, thereby creating a tension bar which supports the neck of the replacement hip.

2. A device as in claim 1 whereby the femoral neck portion of invention articulates with intramedullary portion via press fitting of neck into hole in medial aspect of intramedullary portion.

3. A device as in claim 1 whereby neck portion of invention articulates with intramedullary portion via screw mechanism where neck portion threads into intramedullary portion.

4. A device as in claim 1 whereby neck portion of invention articulates with intramedullary portion via locking mechanism whereby neck portion inserts into intramedullary portion and is then locked via a screw that is tightened within intramedullary portion, advancing screw so that it causes an interference fit against neck of prosthesis, locking it into place.

5. A device as in claim 1 where neck of prosthesis in cross section is either cylindrical or sided, and if sided, those sides being either flat, concave inwardly or convex inwardly.

6. A device as in claim 1 where tension bar articulates with proximal portion of intramedullary portion via fixation with a screw which inserts down into top of intramedullary portion, thereby passing through matching hole in lateral aspect of tension bar and screwing into threads within intramedullary portion thereby securing said tension bar in place.

7. A device as in claim 1 where tension bar articulates with proximal portion of intramedullary portion by sliding down on top of intramedullary portion, whereby tension bar portion has a hollowed part inside of its lateral most part into which a matching shaped proximal portion of intramedullary portion would fit, and through which a screw may or may not be placed, screwing into threads in intramedullary portion.

8. A device as in claim 1 where tension bar articulates with proximal portion of intramedullary portion by the tension bar's lateral most part consisting of a shape which fits into a similar shape in intramedullary portion which is hollow proximally to receive tension bar's shaped end and through which a screw may or may not be placed, screwing into threads in intramedullary portion.

9. A device as in claim 1 where femoral head portion is a continuation of medial aspect of tension bar device and into which the proximal part of the femoral stem can be inserted within a matching hollow cavity within head.

10. A device as in claim 1 where femoral head portion is a continuation of proximal aspect of femoral neck component or articulates with proximal aspect of femoral neck portion via hollow cavity within the base of the femoral head which matches the shape of the proximal aspect of the femoral neck component.

11. A device as in claim 10 where tension bar's medial aspect comprises a ring or hollow structure which encloses the matching shape of the proximal aspect of the femoral neck/head component or femoral neck component.

12. A device as in claim 1 where there is a hole or holes placed distally in the intramedullary portion of device through which interlocking screws can be placed, locking device into bone distally.

13. A device as in claim 1 where there is a threaded screw hole distally on intramedullary device which is oriented along longitudinal axis of intramedullary device and into which can be threaded an end cap or a femoral component of a total knee replacement component if needed.
